SEQUENCE LISTING (9 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 1827 aa, Homo sapiens
MADERKDEGKAPHWTSAPLTEASAHSHPPEIKDQGGAGEGLVRSANGFPYREDEEGAFGEHGSQGTYSNTKENGINGELTSADRETAEEVSARIVQVVTAEAVAVLKGEQEKEAQHKDQTAALPLAAEETANLPPSPPPSPASEQTVTVEEDLLTASKMEFHDQQELTPSTAEPSDQKEKESEKQSKPGEDLKHAALVSQPETTKTYPDKKDMQGTEEEKAPLALFGHTLVASLEDMKQKTEPSLVVPGIDLPKEPPTPKEQKDWFIEMPTEAKKDEWGLVAPISPGPLTPMREKDVFDDIPKWEGKQFDSPMPSPFQGGSFTLPLDVMKNEIVTETSPFAPAFLQPDDKKSLQQTSGPATAKDSFKIEEPHEAKPDKMAEAPPSEAMTLPKDAHIPVVEEHVMGKVLEEEKEAINQETVQQRDTFTPSGQEPILTEKETELKLEEKTTISDKEAVPKESKPPKPADEEIGIIQTSTEHTFSEQKDQEPTTDMLKQDSFPVSLEQAVTDSAMTSKTLEKAMTEPSALIEKSSIQELFEMRVDDKDKIEGVGAATSAELDMPFYEDKSGMSKYFETSALKEEATKSIEPGSDYYELSDTRESVHESIDTMSPMHKNGDKEFQTGKESQPSPPAQEAGYSTLAQSYPSDLPEEPSSPQERMFTIDPKVYGEKRDLHSKNKDDLTLSRSLGLGGRSAIEQRSMSINLPMSCLDSIALGFNFGRGHDLSPLASDILTNTSGSMDEGDDYLPATTPALEKAPCFPVESKEEEQIEKVKATGEESTQAEISCESPFLAKDFYKNGTVMAPDLPEMLDLAGTRSRLASVSADAEVARRKSVPSETVVEDSRTGLPPVTDENHVIVKTDSQLEDLGYCVFNKYTVPLPSPVQDSENLSGESGTFYEGTDDKVRRDLATDLSLIEVKLAAAGRVKDEFSVDKEASAHISGDKSGLSKEFDQEKKANDRLDTVLEKSEEHADSKEHAKKTEEAGDEIETFGLGVTYEQALAKDLSIPTDASSEKAEKGLSSVPEIAEVEPSKKVEQGLDFAVQGQLDVKISDFGQMASGLNIDDRRATELKLEATQDMTPSSKAPQEADAFMGVESGHMKEGTKVSETEVKQKVAKPDLVHQEAVDKEESYESSGEHESLTMESLKADEGKKETSPESSLIQDEIAVKLSVEIPCPPAVSEADLATDERADVQMEFIQGPKEESKETPDISITPSDVAEPLHETIVSEPAEIQSEEEEIEAQGEYDKLLFRSDTLQITDLGVSGAREEFVETCPSEHKGVIESVVTIEDDFITVVQTTTDEGESGSHSVRFAALEQPEVERRPSPHDEEEFEVEEAAEAQAEPKDGSPEAPASPEREEVALSEYKTETYDDYKDETTIDDSIMDADSLWVDTQDDDRSIMTEQLETIPKEEKAEKEARRSSLEKHRKEKPFKTGRGRISTPERKVAKKEPSTVSRDEVRRKKAVYKKAELAKKTEVQAHSPSRKFILKPAIKYTRPTHLSCVKRKTTAAGGESALAPSVFKQAKDKVSDGVTKSPEKRSSLPRPSSILPPRRGVSGDRDENSFSLNSSISSSARRTTRSEPIRRAGKSGTSTPTTPGSTAITPGTPPSYSSRTPGTPGTPSYPRTPHTPGTPKSAILVPSEKKVAIIRTPPKSPATPKQLRLINQPLPDLKNVKSKIGSTDNIKYQPKGGQVQIVTKKIDLSHVTSKCGSLKNIRHRPGGGRVKIESVKLDFKEKVQAKVGSLDNAHHVPGGGNVKIDSQKLNFREHAKARVDHGAEIITQSPGRSSVASPRRLSNVSSSGSINLLESPQLATLAEDVTAALAKQGL

SEQ ID NO: 3 - protein, 16 aa, Artificial Sequence
  Description of Artificial Sequence Synthetic peptide
  MOD_RES (8): PHOSPHORYLATION
CSIDMVDSPQLATLAD

SEQ ID NO: 4 - protein, 15 aa, Homo sapiens
SIDMVDSPQLATLAD

SEQ ID NO: 5 - protein, 15 aa, Homo sapiens
SINLLESPQLATLAE

SEQ ID NO: 6 - protein, 15 aa, Artificial Sequence
  Description of Artificial Sequence Synthetic peptide
  MOD_RES (7): PHOSPHORYLATION
SIDMVDSPQLATLAD

SEQ ID NO: 7 - protein, 15 aa, Artificial Sequence
  Description of Artificial Sequence Synthetic peptide
  MOD_RES (7): PHOSPHORYLATION
SINLLESPQLATLAE

SEQ ID NO: 8 - protein, 15 aa, Artificial Sequence
  Description of Artificial Sequence Synthetic consensus sequence
  MOD_RES (3): Asp or Asn
  MOD_RES (4): Met or Leu
  MOD_RES (5): Val or Leu
  MOD_RES (6): Asp or Glu
  MOD_RES (15): Asp or Glu
SIXXXXSPQLATLAX

SEQ ID NO: 9 - protein, 15 aa, Artificial Sequence
  Description of Artificial Sequence Synthetic consensus sequence
  MOD_RES (3): Asp or Asn
  MOD_RES (4): Met or Leu
  MOD_RES (5): Val or Leu
  MOD_RES (6): Asp or Glu
  MOD_RES (7): PHOSPHORYLATION
  MOD_RES (15): Asp or Glu
SIXXXXSPQLATLAX

(19) United States
(12) Patent Application Publication
(10) Pub. No.: US 2007/0280935 A1
(43) Pub. Date: Dec. 6, 2007

(54) Antibody that recognizes phosphorylated peptides

(76) Inventors: Bernd Bohrmann, Riehen (CH); Christian Czech, Grenzach-Wyhlen (DE); Judith Gerlach-Weck, Zornheim (DE); Fiona Grueninger, Arlesheim (CH)

Correspondence Address: HOFFMANN-LA ROCHE INC.;PATENT LAW DEPARTMENT, 340 KINGSLAND STREET, NUTLEY, NJ, 07110, US

(21) Appl. No.: 11/824,066

(22) Filed: Jun. 29, 2007

(30) Foreign Application Priority Data
Apr. 7, 2006 (EP) 06116550.2

(51) Int. Cl. A61K 39/395; G01N 33/567; C07K 16/40

(52) U.S. Cl. 424/133.1; 435/7.2; 530/388.26

(57) ABSTRACT

The present invention relates to an antibody which recognizes an epitope consisting of Ser-Ile-A1-A2-A3- A4-Ser(PO₃H₂)-Pro-Gln-Leu-Ala-Thr-Leu-Ala-A5 (SEQ ID NO: 9), and does not bind to an epitope consisting of Ser-Ile-A1-A2-A3- A4-Ser-Pro-Gln-Leu-Ala-Thr-Leu-Ala- A5 (SEQ ID NO: 8), wherein A1 is Asp or Asn, A2 is Met or Leu, A3 is Val or Leu, A4 is Asp or Glu and A5 is Asp or Glu, a hybridoma producing the antibody, a kit comprising the antibody, and a method for diagnosing a neurological disorder using the antibody.

### PRIORITY TO RELATED APPLICATION(S)

This application claims the benefit of European Patent Application No. 06116550.2, filed Jul. 4, 2006, which is hereby incorporated by reference in its entirety.

The present invention relates to an antibody which binds to an epitope consisting of Ser-Ile-A1-A2-A3-A4-Ser (PO₃H₂)-Pro-Gln-Leu-Ala-Thr-Leu-Ala-A5, and does not bind to an epitope consisting of Ser-Ile-A1-A2-A3-A4-Ser-Pro-Gln-Leu-Ala-Thr-Leu-Ala-A5, wherein A1 is Asp or Asn, A2 is Met or Leu, A3 is Val or Leu, A4 is Asp or Glu and A5 is Asp or Glu.

### BACKGROUND OF THE INVENTION

Alzheimer's Disease (AD) is the most common form of adult-onset dementia. There is currently no reliable biochemical test or biomarker available for diagnosis of AD and conclusive diagnosis can only be made post-mortem.

Neuropathological examination of post-mortem brain shows large amounts of extracellular amyloid plaques and intracellular neurofibrillary tangles (NFTs) in characteristic regions of the brain. NFT deposition correlates better with disease severity than plaque accumulation.

NFTs comprise paired helical filaments (PHFs) of the microtubule-associated protein tau (Delacourte, A, J., Neurol. Sci. 76 (1986) 173-180, Kosik, K., PNAS 83 (1986) 4044-4048, Kondo, J., Neuron 1 (1988) 82, Wood, J., PNAS 83 (1986) 4040-4043). The function of tau in vivo is to promote microtubule assembly and stability within the axonal compartment of neurons (Lewis, S., Nature 342 (1989) 498-505).

Tau is a highly soluble protein, with no propensity whatsoever to aggregate into filaments. However, in AD, tau becomes hyperphosphorylated. This hyperphosphorylation initiates an aggregation processes which culminates in PHFs and ultimately NFTs. Phosphorylation is a normal feature of tau and is part of the control mechanism which regulates the binding to microtubules (Lovestone, S., Biol. Psychiatry 45(1999) 995-1003).

There is a large number of potential phosphorylation sites within the molecule, these occur within two regions which flank the microtubule binding domain of tau. Phosphorylation at these sites is benign and does not lead to tau aggregation. In AD, a discrete number of additional phosphoepitopes (epitopes comprising at least one phosphorylated amino acid) are found and it is this hyperphosphorylation or abnormal phosphorylation which most likely initiates the fibrillization process. An understanding of the mechanisms that lead to PHF-tau formation therefore requires knowledge of these phosphorylation sites

The abnormal phosphoepitopes in tau that occur in AD have been identified by isolation and characterization of PHFs from AD brain (Hasegawa, M., J. Biol. Chem. 267 (1992) 17047-17054, Cripps, D., J. Biol. Chem. 281 (2006) 10825-10838). One of these phosphoepitopes occurs at Ser422, towards the C-terminus of tau. Phosphorylation of Ser422 is tightly linked to NFT formation (Jicha, G., J. Neurochem 69 (1997) 2087-2095). This group showed in cell culture experiments that phosphorylation of tau at Ser422 causes a decrease in tau solubility, implying a role in the initial steps of the aggregation process. Mutation of Ser422 to alanine abolished this effect (Ferrari, A., J. Biol. Chem. 278 (2003) 40162-40168).

Phosphorylation of tau at Ser422 may be a sensitive marker for AD, as suggested by an early study using a polyclonal antibody directed against this phosphoepitope (Bussiere, T., Acta. Neuropathol. 97 (1999) 221-230). However, a high affinity, highly selective monoclonal antibody has not been available for this phosphoepitope until now. Such an antibody must be capable of recognising tau that is phosphorylated at Ser422 against a high background of non-phosphorylated tau and of tau phosphorylated at other pathogenic and non-pathogenic epitopes. The high degree of selectivity is crucial for allowing the clear distinction of pathological from non-pathological events in AD.

A number of publications point to the importance of the single phosphoepitope, phospho-Ser422, in the development of tau pathology.

In addition, mass spectrometric analysis of tau derived from NFTs or from normal brain showed that this phosphoepitope is unique to PHF-tau (Hasegawa, M., J. Biol. Chem. 267 (1992) 17047-17054, Cripps, D., J. Biol. Chem. 281 (2006) 10825-10838).

There are many phosphoepitopes in PHF-tau (paired helical filament-tau) and several monoclonal antibodies recognizing one or more of these phosphoepitopes are known e.g. TG3 (pThr231), PHF-1 (pSer396/pSer4O4), 12E8 (pS262/pS356), AT8 (pSer2O2/p (pSer212/pThr214), AT180 (pThr231) and AT270 (p181) (Seubert, P., J. Biol.Chem. 270 (1995) 18917-18922, Greenberg, S., J. Biol. Chem. 267 (1992) 564-569, Jicha, G., J.Neurochem. 69 (1997) 2087-2095, Mercken, M., Acta. Neuropathol. 84 (1992) 265-272).

Some of these antibodies, for instance 12E8, AT180 and AT270, also recognize tau from healthy brains and are not truly disease-state specific. All of these antibodies (except 12E8) were raised using purified PHF-tau as an immunogen and consequently the antigenic epitope recognized by these antibodies is complex, comprising either double/multiple phosphorylations or a combination of conformational and phosphoepitope. These antibodies are therefore of limited use in analyzing the contribution of individual phosphoepitopes to the development of tau pathology.

Although many polyclonal antibodies have been raised against individual phosphoepitopes, many of these show some degree of cross-reactivity with normal tau. One monoclonal antibody, AP422, has previously been described which was raised against a tau- derived phosphopeptide containing phospho-Ser422. However, this antibody shows weak cross- reactivity with normal tau (Hasegawa, M., FEBS Letters 384 (1996) 25-30).

In order to assess the contribution of this phosphoepitope to tau pathology, and to develop immunoassays for measuring levels of this phosphotau variant in brain tissues derived from AD patients, a high affinity, highly selective monoclonal antibody showing no cross-reactivity with normal tau is required.

### SUMMARY OF THE INVENTION

The present invention provides an antibody which binds to an epitope consisting of Ser-Ile-A1-A2-A3- A4-Ser(PO₃H₂)-Pro-Gln-Leu-Ala-Thr-Leu-Ala-A5 (SEQ ID NO: 9), and does not bind to an epitope consisting of Ser-Ile-A1-A2-A3- A4-Ser-Pro-Gln-Leu-Ala-Thr-Leu-Ala-A5 (SEQ ID NO:8), wherein A1 is Asp or Asn, A2 is Met or Leu, A3 is Val or Leu, A4 is Asp or Glu and A5 is Asp or Glu. Furthermore, the present invention provides an antibody which binds to tau comprising phospho-Ser422, and does not bind to tau comprising unphosphorylated Ser422. Furthermore, the present invention provides a method for detecting tau comprising phospho-Ser422. Furthermore, the present invention provides use of the antibody for detecting tau comprising phospho Ser-422. Furthermore, the present invention provides a kit for detecting tau comprising phospho-Ser422. Furthermore, the present invention provides a cell which produces the antibody.

### BRIEF DESCRIPTION OF THE DRAWINGS

FIG. 1 shows specificity of the monoclonal antibodies. Specificity of the monoclonal antibodies was tested by western blot analysis of various tau species with 441aa. A: Pan α-tau (control), antibodies 2.5.2 and 2.20.4; B: monoclonal antibody AP422
Lane1:tau comprising unphosphorylated Ser at position 422
Lane2:phosphorylated tau comprising phosphorylated Ser at position 422
Lane3:tau comprising Ser Ala mutation at position 422
Lane4:phosphorylated tau comprising Ser→Ala mutation at position 422

FIG. 2 shows the result of western blot analysis of soluble- and insoluble extracts from Braak-staged AD brain tissue. Several tau bands are visible due to the fact that the human brain contains multiple splicing isoforms of tau. Note that neither Braak stage II/control brain, nor the soluble fraction, contain tau phosphorylated at Ser422. M=lane containing marker proteins (Novex); I=insoluble fraction; S=soluble fraction; II, IV, VI=Disease severity (Braak stage)

FIG. 3 shows the result of Immunohistochemical analysis of AD brain tissue (Braak stage VI). NFT=neurofibrillary tangle; NT=neuropil thread; DN=dystrophic neuritis (surrounding amyloid plaque). Antibody used was 2.5.2. Control brain showed no staining.

FIG. 4 shows the result of western blot analysis of okadaic acid-treated LAN-5 cells (OA) using antibody 2.5.2. The antibody reacted with three proteins which were all phosphoproteins, as indicated by the lack of reactivity with vehicle/DMSO extract or with extract from cells treated with the pan-kinase inhibitor K252a. The individual molecular weights were consistent with the largest protein being MAP 2a/b (phosphorylated at S1808) and the two smaller proteins being two different isoforms of tau (phosphorylated at S422). Lane 1=vehicle; lane 2=OA, lane 3=OA+K252a.

FIG. 5 shows the result of ELISA for determination of tau/pser422 levels in okadaic acid-treated LAN-5 cells. The amount of tau/pSer422 was calculated on the basis of 1×10⁶ cells.

### DETAILED DESCRIPTION OF THE INVENTION

More specifically, the present invention provides the following (1) to (24).
(1) An antibody or fragment thereof which binds to an epitope consisting of Ser-Ile-A1-A2-A3-A4-Ser(PO₃H₂)-Pro-Gln-Leu-Ala-Thr-Leu-Ala-A5 (SEQ ID NO: 9), and does not bind to an epitope consisting of Ser-Ile-Al-A2-A3- A4-Ser-Pro-Gln-Leu-Ala-Thr-Leu-Ala-A5 (SEQ ID NO: 8), wherein A1 is Asp or Asn, A2 is Met or Leu, A3 is Val or Leu, A4 is Asp or Glu and A5 is Asp or Glu.
(2) The antibody of (1), wherein A1 is Asp, A2 is Met, A3 is Val, A4 is Asp and A5 is Asp.
(3) The antibody of (1), wherein A1 is Asn, A2 is Leu, A3 is Leu, A4 is Glu and A5 is Glu.
(4) The antibody of (1) or (2), wherein the antibody binds to tau comprising phospho-Ser422 and does not bind to tau comprising unphosphorylated Ser422.
(5) The antibody of (1) to (4), wherein the antibody binds to MAP2 comprising phospho-Ser1808 and does not bind to MAP2 comprising unphosphorylated Ser1808.
(6) The antibody of (2), wherein its Kd for the peptide consisting of Ser-Ile-Asp-Met-Val-Asp-Ser(PO₃H₂)-Pro-Gln-Leu-Ala-Thr-Leu-Ala-Asp (SEQ ID NO: 6) is lower than 100 nM.
(7) The antibody of (1) to (6), wherein the antibody is monoclonal antibody.
(8) The antibody of (1) to (7), wherein the antibody is produced by the hybridoma DSM ACC2762 or DSM ACC2763.
(9) A method for diagnosing a neurological disorder, which comprises
   (a) contacting a biological sample with the antibody of (1) to (8); and
   (b) detecting a complex formed between the antibody and tau or MAP2.
(10) The method of (9), wherein neurological disorder is Alzheimer's disease.
(11) A method for detecting tau in a biological sample comprising phospho-Ser422, which comprises
   (a) contacting a biological sample with the antibody of (1) to (8); and
   (b) detecting a complex formed between the antibody and tau.
(12) A method for detecting MAP2 in a biological sample comprising phospho-Ser1808, which comprises
   (a) contacting a biological sample with the antibody of (1) to (8); and
   (b) detecting a complex formed between the antibody and MAP2.
(13) The method of (11) or (12), wherein the complex is detected by western blotting, immunohistochemistry or ELISA.
(14) Use of the antibody of (1) to (8) for diagnosis of neurological disorders.
(15) Use of (14), wherein neurological disorder is Alzheimer's disease.
(16) Use of the antibody of (1) to (8) for detecting tau comprising phospho-Ser422.
(17) Use of the antibody of (1) to (8) for detecting MAP2 comprising phospho-Ser1808.
(18) A kit for diagnosis of neurological disorders, which comprises the antibody of (1) to (8).
(19) A kit of (18), wherein neurological disorder is Alzheimer's disease.
(20) A kit for detecting tau comprising phosho-Ser422, which comprises the antibody of (1) to (8).
(21) A kit for detecting MAP2 comprising phospho-Ser1808, which comprises the antibody of (1) to (8).
(22) A cell which produces the antibody of (1) to (8).
(23) The cell of (22), wherein the cell is a hybridoma.
(24) The cell of (23), wherein the hybridoma is DSM ACC2762 or DSM ACC2763.

The present invention provides an antibody which binds to an epitope consisting of Ser-Ile-A1-A2-A3- A4-Ser(PO₃H₂)-Pro-Gln-Leu-Ala-Thr-Leu-Ala-A5 (SEQ ID NO:9), and does not bind to an epitope consisting of Ser-Ile-A1-A2-A3- A4-Ser-Pro-Gln-Leu-Ala-Thr-Leu-Ala-A5 (SEQ ID NO:8), wherein A1 is Asp or Asn, A2 is Met or Leu, A3 is Val or Leu, A4 is Asp or Glu and A5 is Asp or Glu.

In the present invention, “Ser(PO₃H₂)” means phosphorylated serine.

In one embodiment, the antibody of the present invention binds to an epitope consisting of Ser-Ile-Asp-Met-Val-Asp-Ser(PO₃H₂)-Pro-Gln-Leu-Ala-Thr-Leu-Ala-Asp (SEQ ID NO:6), and does not bind to an epitope consisting of Ser-Ile-Asp-Met-Val-Asp-Ser-Pro-Gln-Leu-Ala-Thr-Leu-Ala-Asp (SEQ ID NO: 4).

In the preferred embodiment, the present invention provides the antibody which binds to phosphorylated tau comprising phospho-Ser422, and does not bind to tau comprising unphosphorylated Ser422.

There are several splicing isoforms of tau, such as, Fetal-tau, Tau-A, Tau-B, Tau-C, Tau D, Tau-E, Tau-F (Swiss-Prot; Entry name:TAU_HUMAN, Primary accession number:P10636). In the present invention, preferably, tau is a polypeptide having the sequence of SEQ ID NO: 1. The term “tau” also contains splicing isoforms, variant, derivatives, homologues or fragments of the polypeptide having the sequence of SEQ ID NO:1. In the case tau is splicing isoform, variant, derivative, homologue or fragment of the polypeptide having the sequence of SEQ ID NO:1, it is preferable that tau comprises the sequence of Ser-Ile-Asp-Met-Val-Asp-Ser-Pro-Gln-Leu-Ala-Thr-Leu-Ala-Asp (SEQ ID NO:4).

The term “phosphorylated tau” refers to tau in which at least one amino acid is phosphorylated.

The term “phospho-Ser422” refers to a phosphorylated Serine at position 422 as defined by the position in the amino acid sequence of SEQ ID NO:1. The term “phospho-Ser422” also contains a phosphorylated Serine in splicing isoform, derivatiw, variant, homologue or fragment, which is corresponding to the phosphorylated Serine at position 422 of the polypeptide having the sequence of SEQ ID NO:1.

It is preferable that the antibody of the present invention has high binding affinity to tau comprising phospho-Ser422. The term “high binding affinity”, as the term is used herein, means dissociation constant K_{d} of an antibody for tau comprising phospho-Ser422 is lower than 100 nM. K_{d} of an antibody can be determined by methods known to those skilled in the art. For example, K_{d} can be determined using surface plasmon resonance with Biacore under the condition described in the example 4. To use the peptide consisting of Ser-Ile-Asp-Met-Val-Asp-Ser (PO₃H₂)-Pro-Gln-Leu-Ala-Thr-Leu-Ala-Asp (SEQ ID NO:6) for determining K_{d} is preferable.

In one embodiment, the antibody of the present invention binds to an epitope consisting of Ser-Ile-Asn-Leu-Leu-Glu-Ser(PO₃H₂)-Pro-Gln-Leu-Ala-Thr-Leu-Ala-Glu (SEQ ID NO:7), and does not bind to an epitope consisting of Ser-Ile-Asn-Leu-Leu-Glu-Ser-Pro-Gln-Leu-Ala-Thr-Leu-Ala-Glu (SEQ ID NO:5).

In a preferred embodiment, the antibody of the present invention binds to MAP2 (microtubule-associated protein 2) comprising phospho-Ser1808, and does not bind to MAP2 comprising unphosphorylated Ser1808.

There are a few isoforms of MAP2, such as isoform1, isoform2 or isoform3 (Swiss-Prot; Entry name:MAP2_HUMAN, Primary accession number:P11137). In the present invention, preferably, MAP2 is a polypeptide having the sequence of SEQ ID NO:2. The term “MAP2” also contains splicing isoform, variant, derivative, homologue or fragment of the polypeptide having the sequence of SEQ ID NO:2. In the case MAP2 is splicing isoform, variant, derivative, homologue or fragment of the polypeptide having the sequence of SEQ ID NO:2, it is preferable that MAP2 has the sequence of Ser-Ile-Asn-Leu-Leu-Glu-Ser-Pro-Gln-Leu-Ala-Thr-Leu-Ala-Glu (SEQ ID NO:5).

The term “phosphorylated MAP2” refers to MAP2 in which at least one amino acid is phosphorylated.

The term “phospho-Ser1808” refers to a phosphorylated Serine at position 1808 as defined by the position in the amino acid sequence of SEQ ID NO:2. The term “phospho-Ser 1808” also contains a phosphorylated Serine in splicing isoform, derivative, variant, homologue or fragment, which is corresponding to the phosphorylated Serine at position 1808 of the polypeptide having the sequence of SEQ ID NO:2.

Whether an antibody binds to the epitope or not can be determined by well known methods, such as western blot, enzyme immunoassay or surface plasma resonance. A polypeptide consisting of the sequence of the epitope, a whole protein comprising the sequence of the epitope or a fragment of the protein can be used for detecting the binding.

The antibody of the present invention may be polyclonal or monoclonal antibody. In a preferable embodiment, the antibody of the present invention is a monoclonal antibody.

The term “monoclonal antibody” refers to an antibody obtained from a group of substantially homogeneous antibodies, i.e., antibody group wherein the antibodies constituting the group are homogeneous except for naturally occurring mutants that exist in a small amount.

In a preferred embodiment, the antibody of the present invention is the antibody produced by the hybridoma MAK<pTAU>M.2.5.2 or MAK<pTAU>M.2.20.4. The hybridoma MAK<pTAU>M.2.5.2 was deposited at DSMZ (Deutsche Sammlung von Mikroorganismen und Zellkulturen GmBH, Mascheroderweg1b, D-38124 Braunschweig, Germany) on Mar. 15, 2006 under No. DSM ACC2762. The hybridoma MAK<pTAU>M.2.20.4 was deposited at DSMZ (Deutsche Sammlung von Mikroorganismen und Zellkulturen GmBH, Mascheroderweg1b, D-38124 Braunschweig, Germany) on Mar. 15, 2006 under No. DSM ACC2763.

The antibody produced by the hybridoma DSM ACC2762 (MAK<pTAU>M.2.5.2) or DSM ACC2763 (MAK<pTAU>M.2.20.4) has high affinity and high selectivity with tau comprising phospho-Ser422, and does not have cross-reactivity with tau comprising unphosphorylated Ser422. Additionally, the antibody produced by the hybridoma binds to MAP2 comprising phospho-Ser1808, and does not bind to MAP2 comprising unphosphorylated Ser1808. These antibodies can be used for detecting tau comprising phospho-Ser422 or MAP2 comprising phospho-Ser1808. The antibody produced by the hybridoma DSM ACC2762 or DSM ACC2763 is suitable for diagnosis of neurological diseases such as Alzheimer's disease. The antibody produced by the hybridoma DSM ACC2762 or DSM ACC2763 is also suitable for detecting tau comprising phospho-Ser422 or MAP2 comprising phospho-Ser1808.

The antibody of the present invention can be obtained as a polyclonal antibody or a monoclonal antibody, using known techniques. For example, the monoclonal antibody can be produced by the hybridoma method (Kohler G. and Milstein C., Nature 256 (1975) 495-497) or the recombinant method (U.S. Pat. No. 4,816,567). The monoclonal antibody can be also isolated from aphage antibody library (Clackson T., Nature 352(1991) 624-628).

Hybridoma producing the antibody can be prepared essentially using known techniques. For example, following methods can be used: An animal is immunized by a conventional immunization method using a sensitizing antigen to obtain an immune cell, which is then fused to a known parent cell by a conventional cell fusion method. Fused cells are screened for monoclonal antibody-generating cells by a conventional screening method. More specifically, the monoclonal antibody can be obtained as follows.

First, a fragment which has the sequence of Ser-Ile-A1-A2-A3- A4-Ser-Pro-Gln-Leu-Ala- Thr-Leu-Ala-A5 (SEQ ID NO: 8) (A1 is Asp or Asn, A2 is Met or Leu, A3 is Val or Leu, A4 is Asp or Glu and A5 is Asp or Glu), for use as a sensitized antigen is prepared. The fragment can be prepared by chemical synthesis or culturing host cell comprising a gene coding the fragment. Then, the fragment is phosphorylated bykinase, for example ERK2 kinase. A whole protein comprising the fragment may also be used as a sensitized antigen. In the present invention, preferable sensitized antigen is the phosphorylated fragment of tau, which has the sequence of Ser-Ile-Asp-Met-Val-Asp-Ser(PO₃H₂)-Pro-Gln-Leu-Ala-Thr-Leu-Ala-Asp (SEQ ID NO:6).

There is no limitation as to the type of mammalian species to be immunized with the sensitized antigen. However, a mammal is preferably selected based on its compatibility with the parental cell to be used in cell fusion. Generally, rodents such as for example mice, rats, hamsters or rabbits, and monkeys can be used.

For immunization of animals with a sensitizing antigen, known methods may be employed.

Animals can be immunized with a sensitized antigen by well known methods such as a routine method of injecting a sensitized antigen into a mammal intraperitoneally or subcutaneously. Specifically, the sensitized antigen is diluted appropriately with phosphate-buffered saline (PBS), physiological saline and such, and then suspended. An adequate amount of a conventional adjuvant, for example, Freund's complete adjuvant, is mixed with the suspension, as necessary. An emulsion is then prepared for administering to a mammal several times over a 4-to 21-day interval. An appropriate carrier may be used for the sensitized antigen in immunization.

A mammal is immunized as described above. After a titer increase of target antibody in the serum is confirmed, immunocytes are collected from the mammal and then subjected to cell fusion. Spleen cells are the preferred immunocytes.

Mammalian myeloma cells are used as the parental cells to be fused with the above immunocytes. Preferable myeloma cells to be used include various known cell lines, for example, P3(P3x63Ag8.653) (Kearney J F, et al., J. Immnol. 123, 1548-1550 (1979)), P3x63Ag8U. 1 (yelton DE, et al., Current Topics in Microbiology and Immunology 81, 1-7 (1978)), NS-1 (Kohler, G. and Milstein, C. Eur. J. Immunol. 6, 511-519 (1976)), MPC-11 (Margulies, D. H. et al., Cell 8, 405-415 (1976)), SP2/0 (Shulman, M. et al., Nature 276, 269-270 (1978)), FO (deSt. Groth, S. F. et al., J. Immunol. Methods 35, 1-21 (1980) ), S194 (Trowbridge, I. S., J. Exp. Med. 148, 313-323 (1978)), and R210 (Galfre, G. et al., Nature 277, 131-133 (1979)).

Cell fusions between the immunocytes and the myeloma cells as described above can be essentially carried out using methods known to a person skilled in the art, for example, a method by Kohler and Milstein (Kohler, G. and Milstein, C., Methods Enzymol. 73: 3-46 (1981)).

More specifically, the above-described cell fusions may be carried out, for example, in a conventional culture medium in the presence of a cell fusion- promoting agent. The fusion-promoting agents include but are not limited to polyethylene glycol (PEG) and Sendai virus (HVJ). If required, an auxiliary substance such as dimethyl sulfoxide may also be added to improve fusion efficiency.

The ratio of immunocytes to myeloma cells may be determined at one's own discretion, preferably, one myeloma cell for every one to ten immunocytes. Culture media to be used for the above cell fusions include, for example, media that are suitable for the growth of the above myeloma cell lines, such as RPMI 1640 media and MEM media, and other conventional culture media used for this type of cell culture. In addition, serum supplements such as fetal calf serum (FCS) may also be used in combination.

Cell fusion is carried out as follows. As described above, predetermined amounts of immunocytes and myeloma cells are mixed well in the culture medium. PEG solution (for example, mean molecular weight of about 1,000-6,000) pre-heated to 37° C. is added to the cell suspension typically at a concentration of 30% to 60% (w/v), and mixed to produce fused cells (hybridomas). Then, an appropriate culture medium is successively added to the mixture, and the sample is centrifuged to remove supernatant. This treatment is repeated several times to remove the unwanted cell fusion-promoting agent and others that are unfavorable to hybridoma growth.

Screening of the resulting hybridomas can be carried out by culturing them in a conventional selective medium, for example, hypoxanthine, aminopterin, and thymidine (HAT) medium. Culturing in the above-descried HAT medium is continued for a period long enough (typically, for several days to several weeks) to kill cells (non-fused cells) other than the desired hybridomas. Then, hybridomas are screened for single-cell clones capable of producing the target antibody by conventional limiting dilution methods.

In addition to the method for preparing the above-described hybridomas by immunizing non-human animals with antigens, human antibodies can also be obtained by sensitizing human lymphocytes with a sensitized antigen in vitro; and fusing the sensitized lymphocytes with human myeloma cells capable of dividing permanently (see, for example, Japanese Patent Application No. (JP-B) Hei 1-59878).

Alternatively, it is possible to obtain human antibodies from immortalized cells producing the antibodies. In this method, the cells producing the antibodies are prepared by administering a sensitized antigen to transgenic animals comprising a repertoire of the entire human antibody genes (see for example WO 94/25585, WO 93/12227, WO 92/03918, and WO 94/02602).

The monoclonal antibody-producing hybridomas thus prepared can be passaged in a conventional culture medium, and stored in liquid nitrogen over long periods of time.

Monoclonal antibodies can be prepared from the above-described hybridomas by, for example, a routine procedure of culturing the hybridomas and obtaining antibodies from the culture supernatants.

Once monoclonal antibody is obtained from a hybridoma, recombinant antibody can also be prepared by conventional methods.

The antibody of the present invention may be a labelled antibody. For the labelling of the antibody, known labels such as radioactive substance (for example, ³²p, ¹²⁵I, ³H, ¹³¹I, ¹⁴C), fluorochrome (for example fluorescein, rhodamine, umbeliferone), enzyme (for example luciferases, oxidase, allakine phosphatase, β-galactosidase, β-glucosidase, lysozyme, glucoamylase), coenzyme (for example biotin) or chemiluminescence substance can be used. The labeling of the antibody can be done by well known methods, such as for example glutaraldehyde method, maleimide method, pyridyl disulfide method or periodic acid method.

The antibody of the present invention may also be fragment of the antibody as long as the fragment can bind to an epitope consisting of Ser-Ile-A1-A2-A3- A4-Ser (PO₃H₂)-Pro-Gln-Leu-Ala-Thr-Leu-Ala-A5 (SEQ ID NO:9), and does not bind to an epitope consisting of Ser-Ile-A1-A2-A3- A4-Ser-Pro-Gln-Leu-Ala-Thr-Leu-Ala-A5 (SEQ ID NO:8) (A1 is Asp or Asn, A2 is Met or Leu, A3 is Val or Leu, A4 is Asp or Glu and A5 is Asp or Glu). The fragment of antibody may be, but not limited, Fab, Fab′, Fv, F(ab′)2, scFv, sc(Fv)2 or diabody.

Preferably, the antibody of the present invention forms an immunological complex with tau or fragment thereof comprising a phosphorylated Ser at position 422 as defined by the position of the amino acid in SEQ ID NO:1. More preferably, said tau or fragment thereof is isolated from autopsy-derived brain tissue of AD patients or patients having any other neurological disorders with NFT present. Preferably, the antibody of the present invention does not form such an immunological complex with autopsy-derived brain material from patients having died or suffering from other diseases without the occurrence of NFTs in the brain.

The antibody of the present invention can be used for diagnosis of a neurological disorder such as Alzheimer's disease by detecting phosphorylated polypeptide. The antibody of the present invention can be also used for detection of phosphorylated polypeptide comprising the sequence of Ser-Ile-A1-A2-A3- A4-Ser(PO₃H₂)-Pro-Gln-Leu-Ala-Thr-Leu-Ala-A5 (SEQ ID NO:9), wherein A1 is Asp or Asn, A2 is Met or Leu, A3 is Val or Leu, A4 is Asp or Glu and A5 is Asp or Glu, such as phosphorylated tau comprising phospho-Ser422 or phosphorylated MAP2 comprising phosho-Ser1808.

Moreover, the present invention provides a cell which produces the antibody of the present invention. The cell of the present invention may be a hybridoma or a recombinant cell. The hybridoma which produces the antibody of the present invention can be obtained by conventional methods described above. In a preferable embodiment, hybridoma of the present invention is hybridoma DSM ACC2762 or DSM ACC2763. The hybridoma DSM ACC2762 was deposited at DSMZ (Deutsche Sammlung von Mikroorganismen und Zellkulturen GmBH, Mascheroderweg 1b, D-38124 Braunschweig, Germany) on Mar. 15, 2006 under No. DSM ACC2762. The hybridoma DSM ACC2763 was deposited at DSMZ (Deutsche Sammlung von Mikroorganismen und Zellkulturen GmBH, Mascheroderweg 1b, D-38124 Braunschweig, Germany) on Mar. 15, 2006 under No. DSM ACC2763.

A recombinant cell of the invention can be obtained by well known methods. Specifically, such recombinant cell can be obtained as follows.

An mRNA encoding the variable (V) region of the antibody which binds to an epitope consisting of Ser-Ile-A1-A2-A3- A4-Ser(PO₃H₂)-Pro-Gln-Leu-Ala-Thr-Leu-Ala-A5 (SEQ ID NO: 9), and does not bind to an epitope consisting of Ser-Ile-A1-A2-A3- A4-Ser-Pro-Gln-Leu-Ala-Thr-Leu-Ala-A5 (SEQ ID NO: 8), wherein A1 is Asp or Asn, A2 is Met or Leu, A3 is Val or Leu, A4 is Asp or Glu and A5 is Asp or Glu, is isolated from hybridomas producing such an antibody.

For mRNA isolation, total RNAs are first prepared by conventional methods such as guanidine ultracentrifugation methods (Chirgwin, J. M. et al., Biochemistry 18, 5294-5299 (1979)), or acid guanidinium thiocyanate-phenol-chloroform (AGPC) methods (Chomczynski, P. et al., Anal. Biochem. 162, 156-159 (1987)), and then the target mRNA is prepared using an mRNA Purification Kit (Pharmacia) and such. Alternatively, the mRNA can be directly prepared using the QuickPrep mRNA Purification Kit (Pharmacia).

A cDNA of the antibody V region is synthesized from the resulting mRNA using reverse transcriptase. cDNA synthesis is carried out using the AMV Reverse Transcriptase First-strand cDNA Synthesis Kit (Seikagaku Co.), or such. Alternatively, cDNA can be synthesized and amplified by the 5′-RACE method (Frohman, M. A. et al., Proc. Natl. Acad. Sci. USA 85, 8998-9002 (1988); Belyavsky, A. et al., Nucleic Acids Res. 17, 2919-2932 (1989)) using the 5′- Ampli FINDER RACE Kit (Clontech) and PCR.

Target DNA fragments are purified from the obtained PCR products and then ligated with vector DNAs to prepare recombinant vectors. The vectors may be introduced into E. coli and such, and colonies are selected for preparing the recombinant vector of interest. The target DNA nucleotide sequence is then confirmed by conventional methods such as the dideoxynucleotide chain termination method.

Once a DNA encoding the V region of target antibody is obtained, the DNA is inserted into an expression vector which comprises a DNA encoding the constant region (C region) of a desired antibody.

The method for obtaining the cells which produce the antibody typically comprises the steps of: inserting the antibody gene into an expression vector, so that the gene is expressed under the regulation of expression regulatory regions, such as enhancer and promotor; and transforming host cells with the resulting vectors to express the antibody. Polynucleotide encoding H chain and L chain, respectively, can be inserted into separate expression vectors and co- transfected into a host cell. Alternatively, polynucleotides encoding both H chain and L chain can be inserted into a single expression vector and transfected into a host cell (see for example WO 94/11523). The host cells are not limited as long as the cells can produce the antibody. For example, the cells may be CHO, COS, NIH3T3, myeloma, BHK, Hela, Vero, amphibian cells, insect cells, plants cells or bacterial cells such as E. coli.

Furthermore, the present invention provides a method for diagnosing a neurological disorder. The method for diagnosing a neurological disorder comprises:(a) contacting a biological sample with the antibody of the present invention, and (b) detecting the complex formed between the antibody and tau or MAP2.

The present invention further provides a method for detecting tau comprising phospho-Ser 422 in a biological sample. The method for detecting tau comprising phospho-Ser422 comprises (a) contacting a biological sample with the antibody of the present invention; and (b) detecting a complex formed between the antibody and tau.

The present invention further provides a method for detecting MAP2 comprising phospho-Ser1808 in a biological sample. The method for detecting MAP2 comprising phospho-Ser 1808 comprises (a) contacting a biological sample with the antibody of the present invention; and (b) detecting a complex formed between the antibody and MAP2.

The term “neurological disorder”, as used herein, refers to a disease that is related to tau comprising phospho-Ser422 or MAP2 comprising phospho-Ser1808, such as diseases caused by tau comprising phospho-Ser422 or MAP2 comprising phospho-Ser180, or diseases of which tau comprising phospho-Ser422 or MAP2 comprising phospho-Ser1808 is detected from a patient.

The term “neurological disorder” includes, but is not limited to, Alzheimer's disease, Down's Syndrome, Pick's Disease, progressive supranuclear palsy and corticobasal degeneration. Although a sample used in the methods of the present invention are not limited as long as there is a possibility the sample comprises tau or MAP2, a biological sample obtained from human is preferred. A biological sample includes, but is not limited to, cerebrospinal fluid, serum, blood, tissue (for example, brain tissue) and cell (for example, brain cell).

A contact of a biological sample with the antibody of the present invention can be achieved under conditions which allow the formation of a complex formed between the antibody and tau or a complex formed between the antibody and MAP2. Such conditions are well known to a person skilled in the art.

The detection of a complex formed between the antibody and tau or a complex formed between the antibody and MAP2 can be carried out by conventional manner, such as western blot, enzyme immunoassay such as enzyme-linked immunosorbant assay(ELISA), radioimmunoassay, fluorescent immunoassay, luminescent immunoassay, immunoprecipitation, immunostaining, immunodiffusion and surface plasma resonance biosensor (such as BIAcore).

The method of the present invention can be carried out in vitro or in vivo, however, in vitro method is preferable.

The present invention also provides use of the antibody of the present invention for diagnosis of neurological disorders, such as Alzheimer's disease. The present invention also provides use of the antibody of the present invention for detecting a phosphorylated polypeptide such as tau comprising phospho-Ser422 or MAP2 comprising phospho-Ser1808.

The antibody of the present invention may be used in vivo or in vitro. In the preferred embodiment, the antibody of the present invention is used in vitro.

The present invention provides a kit comprising the antibody which binds to an epitope consisting of Ser-Ile-A1-A2-A3- A4-Ser(PO₃H₂)-Pro-Gln-Leu-Ala-Thr-Leu-Ala-A5 (SEQ ID NO: 9), and does not bind to an epitope consisting of Ser-Ile-A1-A2-A3- A4-Ser-Pro-Gln-Leu-Ala-Thr-Leu-Ala-A 5 (SEQ ID NO: 8), wherein A1 is Asp or Asn, A2 is Met or Leu, A3 is Val or Leu, A4 is Asp or Glu and A5 is Asp or Glu. This kit can be used, for example, for diagnosis of neurological disorders, detecting tau comprising phospho-Ser422 or detecting MAP2 comprising phospho-Ser1808.

The kit may comprise a carrier, such as insoluble polysaccharide(for example, agarose or cellulose), synthetic resin(for example, polystyrene resin, silicon resin, polyacrylamide resin, polycarbonate resin or nylon resin) or insoluble support(for example, glass). The antibody may be immobilized to the carrier.

The kit may comprise a reagent, such as reaction liquid, blocking solution, or other reagent used in immunoassay.

Having now generally described this invention, the same will become better understood by reference to the specific examples, which are included herein for purpose of illustration only and are not intended to be limiting unless otherwise specified.

### EXAMPLES

The following examples serve merely to illustrate certain aspects of the present invention and should not be viewed as limiting the present invention in scope.

### Example 1

### Generation of Monoclonal Antibodies

A. Immunization of Mice

Mice were immunized with a phosphopeptide comprising the following amino acid sequence Cys-Ser-Ile-Asp-Met-Val-Asp-Ser(PO₃H₂)-Pro-Gln-Leu-Ala-Thr-Leu-Ala-Asp (SEQ ID NO:3) which corresponds to amino acids 415-430 of the longest isoform of human tau (peptide synthesized by NeoMPS, Strasbourg). The naturally occurring Asp 415 was replaced by Cys to allow directed coupling via the thiol to KLH.

10-12-week old female Balb/c (Jackson Laboratory, stock#001026) and NMRI mice (Jackson Laboratory, stock#003076) were injected intraperitoneally with 100 μg of phosphopeptide in Complete Freund's Adjuvant. The mice received three further interperitoneal injections of 100 μg peptide in Freund's Incomplete Adjuvant at monthly intervals. Final immunizations were made at 3, 2 and 1 day before spleen removal by intravenous injection of 50 μg peptide in PBS.

B. Fusion and Cloning

Fusion of the spleen cells from immunized mice was carried out according to Galfré and Milstein, Methods in Enzymology, 73 (1981) 3-46. Thus 1×10⁸ spleen cells from each immunized mouse were mixed with 2×10⁷ myeloma cells (P3X63-Ag8-653, ATCC CRL1580) and centrifuged at 300×g for 10 min at RT. The cells were then washed once with RPMI- 1640 medium (without FCS) and centrifuged once more at 300×g for 10 min in a conical tube. The cells were loosened by tapping the tube and warmed to 37° C. in a pre-heated waterbath . Within the space of 1 minute, 1 ml of PEG was added (polyethylenglycol with molecular weight 1500, Roche Diagnostics). Next, 5 ml of RPMI-1640 medium (without FCS) was added dropwise with gentle shaking and finally the volume was adjusted to 50 ml by addition of RPMI-1640 medium containing 10% FCS. The cell suspension was then centrifuged at 300 ×g for 10 min. The cell pellet was resuspended in RPMI-1640 medium containing 10% FCS and seeded into hypoxanthine-azaserine selection medium (100 mM hypoxanthine [Sigma], 1 μg/ml azaserine [Sigma] in RPMI-1640+10% FCS). The medium was supplemented with 50U/ml interleukin-6 (Roche Applied Science).

After 10 days the primary cultures were tested for specific antibody production (see Example 2). Primary cultures which showed a positive reaction with phosphopeptide and no reaction with unphosphorylated peptide were cloned by fluorescent-activated cell sorting in 96-well cell culture plates. The medium used here was RPMI-1640 medium supplemented with 10%FCS and 25 U/ml interleukin-6.

Two of the hybridoma cell lines/clones obtained in this way were deposited at DSMZ (Deutsche Sammlung von Mikroorganismen und Zellkulturen GmbH, Mascheroder Weg 1b, D-38124 Braunschweig). Table 1 shows the details of the hybridoma.

TABLE 1

| | Deposited at DSMZ on 15 Mar. 2006 | |
|---|---|---|
| Clone | under the following number | IgG Subclass |
| 2.5.2 | DSM ACC2762 | Ig G2a; κ-light chain |
| 2.20.4 | DSM ACC2763 | Ig G2a; κ-light chain |

C. Antibody Purification From Cell Culture Supernatant

Hybridoma cells thus obtained were seeded at a density of 1×10⁵ cells/ml in RPMI-1640 medium supplemented with 10% FCS and 25U/ml interleukin-6 and grown to a cell density of about 3 ×10⁵/ml. The cells were then diluted to 1×10⁶/ml in a final volume of 250 ml and allowed to grow to maximal cell density. The cells were then removed by centrifugation. The hybridoma culture supernatants typically contained ca. 40-50 μg/ml antibody.

Each antibody was purified as follows. Cell-free hybridoma culture supernatant (250-300 ml) was loaded on a 25 ml MEP HyperCel column (Pall Biosciences) which was equilibrated with 50 mM TrisCl pH 8.0. After washing with equilibration buffer the antibody was eluted with 30 mM sodium citrate/100 mM NaCl pH 4.1. Antibody containing fractions were pooled and then dialysed (Spectra/Por 6-8000) overnight at 4° C. against 5 l of SourceQ buffer A. The dialysed MEP pool was loaded on a 10 ml Source 15Q column (GE Healthcare) which was equilibrated with 10 mM TrisCl pH 8.0 (buffer A). After washing with buffer A the antibody was eluted with a gradient from 0-25% buffer B in 10 column volumes. Buffer B contained 10 mM TrisCl/1 M NaCl pH 8.0. The antibody eluted at about 200 mM NaCl. The purity of the individual fractions was checked by SDS-PAGE and the purest fractions pooled. The yield of antibody thus purified was ca. 10 mg/250 ml hybridoma culture supernatant.

### Example 2

### Screening ofr anto-Taqu/pSer422 specific antibodies

A. Determination of Specificity of Antibodies for Phosphopeptide (tau 416-430/pSer422)

In order to measure the specificity of the antibodies in the cell culture supernatants, streptavidin-coated microtiter plates (MicroCoat, Bernried, DE) were coated with 0.1 μg/ml biotinylated phosphopeptide (tau 416-430/pSer422) in PBS, 0.5% Byco C (100 μl/well, 1 h incubation at R.T. with shaking). The plates were then washed 3 times with wash buffer (0.9% NaCl/0.05% Tween 20). Next, 100 of antibody-containing culture supernatant was added in each well and the plates incubated for 1 h at room temperature (R.T.) with shaking. The plates were then washed 3 times with wash buffer. In order to detect bound antibody, 100 μl/well of a polyclonal anti-mouse antibody/peroxidase conjugate (Dianova) for 1 h at R.T. The plates were subsequently washed again. Finally, 100 μl/well of ABTS solution (Roche Diagnostics) was added and the plates incubated for 20 min at R.T. The plates were then read at 405 nm in an X Read Plus microplate reader (Tecan).

B. Measurement of Antibody Cross-reactivity with Non-phosphorylated Peptide (tau 416-430)

The same procedure as described above was used, except that the microtitre plates were coated with non-phosphorylated peptide (tau 416-430).

C. Measurement of Binding to Free Phosphopeptide (tau 416-430/pSer422)

100 μl/well of each cell culture supernatant was pipetted into a microtitreplate that had previously been coated with anti-mouse Fcγ antibody (MicroCoat, Bernried, DE). The plate was then incubated for 1 h at R.T. with shaking and subsequently washed 3 times with wash buffer. Next, 100 μl/well of 50 nM biotinylated phosphopeptide (tau 416-430/pSer422) was added and the plate incubated for 1 h at R.T. with shaking. The plate was washed three times and then incubated with 100 μl/well of 50U/ml streptavidin/peroxidae conjugate (Dianova) for 1 h at R.T. The plate was washed once again and then developed with 100/μl well ABTS solution (Roche Applied Science) for 20 min at R.T. The plate was then read at 405 nm in a X Read Plus microplate reader.

D. Measurement of Antibody Binding to Free Non-phosphorylated Peptide (tau 416-430)

The same procedure as described above was used, except that the microtitre plates were coated with non-phosphorylated peptide (tau 416-430).

Using the methods described above, both of the deposited antibodies showed good binding to immobilized and free phosphopeptide (tau 416-430/pSer422). No cross-reactivity was observed with non-phosphorylated peptide (tau 416-430).

### Example 3

### Determination of antibody specificity towards full-length phosphorylated tau protein and phosphorylated mutant tau protein (S422A) by wester blot

Antibodies were tested for their ability to recognise each of four different tau species, namely tau, tauS422A (Ser→Ala mutation at position 422), p-tau (phosphorylated tau) and p-tauS422A (phosphorylated tau comprising Ser→Ala mutation at position 422). Tau and tauS422A were expressed in E. coli and purified according to standard method. Both proteins were then phosphorylated by ERK2 kinase, which was shown to introduce a phosphogroup at S422 as well as a number of other sites in tau. SDS-PAGE gel was loaded with 150 ng of each of the four tau species; subsequent to electrophoresis the proteins were transferred to nitrocellulose by standard western blotting protocol. Blots were incubated overnight at 4° C. with individual hybridoma cell culture supernatants that had been diluted two-fold with StartingBlock (Perbio). After a standard washing procedure, the blots were incubated in 2 ng/ml anti-mouse antibody/horseradish peroxidase conjugate (Perbio) for 1 hr at RT. The blots were then washed and developed with LumiLight ECL substrate (Roche Applied Science). As shown in FIG. 1A, antibodies 2.5.2 and 2.20.4 (table 1) showed no cross-reactivity with non-phosphorylated tau nor with tau phosphorylated at Ser (or Thr) residues other than Ser422. The antibodies are thus highly selective for the target phosphoepitope. FIG. 1B shows the specificity of the monoclonal antibody AP422. The monoclonal antibody AP422 was obtained from Prof. M. Hasegawa (University of Tokyo, Japan) and diluted 1:2000 with 1% milk in TBSt buffer. The blot was incubated overnight at 4° C. The following day it was washed and incubated for 1 hr at room temperature with anti-mouse/horseradish peroxidase (Pierce) diluted 1:5000 with 1% milk in TBSt buffer. Detection was with the Roche ECL system. The antibody AP422 showed cross-reactivity with unphosphorylated tau.

### Example 4

### Determinatio of kₒₙ, k_{off}, Kₐ and K_{d}

The rate constants kₒₙ and k_{off} and the resulting dissociation constant K_{d} were determined using surface plama resonance (BIAcore 2000 from BIAcore AB).

NHS/EDC-activated Sensorchips (CM5, BIAcore AB) were coated with rabbit-anti mouse IgG at a concentration of 15 μg/ml in 10 mM sodium acetate-acetic acid pH5.0 for 5 min at a flow rate of 20 μl/min. Cell culture supernatants were diluted to a final antibody concentration of 50 nM in 10 mM HEPES pH 7.4, 150 mM NaCl, 3.4 mM EDTA, 0.05% Polysorbate and injected at a flow rate of 10 μl/min over a period of 2 min. Phosphopeptide or non-phosphorylated peptide (0-1000 nM) in 10 mM HEPES pH 7.4,150 mM NaCl, 3.4 mM EDTA, 0.05% Polysorbate was then injected at a flow rate of 100 μl/min for 2 min. This was followed by dissociation for 5 min in 10 mM HEPES pH 7.4, 150 mM NaCl, 3.4 mM EDTA, 0.05% Polysorbate. kₒₙ and k_{off} were then calculated from the sensogramafter double referencing using BIAcore evaluation software (version 4.1, BIAcore AB). Models were fitted gobally across the data sets using the 1:1 (Langmuir) binding interaction. The association constant Kₐ was calculated from kₒₙ/k_{off}

The values obtained are summarized below. Both antibodies have K_{d} values in the low nanomolar range and thus can be considered to bind pSer422 with high affinity. Neither of the antibodies showed any interaction with the non-phosphorylated peptide, indicating that they are highly selective for the phosphoepitope and confirming the western blot results in example3. Table 2 shows Kₙ, K_{off}, Kₐ and K_{d} of the antibody 2.5.2 and the antibody 2.20.4.

TABLE 2

Tau 416-430/pSer422

| | kₒₙ | k_{off} | Kₐ | K_{d} |
|---|---|---|---|---|
| Clone | 1/Ms | 1/s | 1/M | nM |
| 2.5.2 | 8 × 10⁵ | 8 × 10⁻³ | 9 × 10⁷ | 11 |
| 2.20.4 | 7 × 10⁵ | 1 × 10⁻² | 7 × 10⁷ | 14 |

### Example 5

Western Blots of AD Brain Extracts

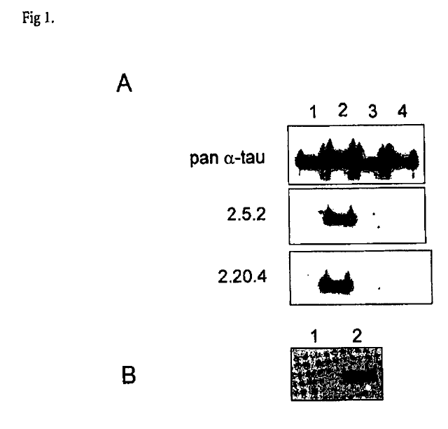

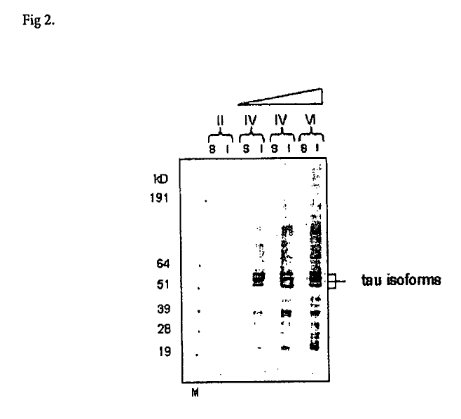

Microtome sections were prepared from Braak-staged AD brains and control brains. Ca. 50 mg wet weight tissue from each brain was dispensed into Eppendorf tubes. Each tissue was then homogenized in 10 volumes of ice-cold RAB-HS buffer using a hand-held glass-homogenizer. Homogenates were then centrifuged at 50000xg for 40 min at 4° C. The resulting pellets were resuspended in Tris-sucrose-SDS by homogenization and centrifuged at 50000xg for 40 min at RT. The resulting supernatants were retained for analysis. SDS-PAGE gel was loaded with 4.5 μl each supernatant. Western blotting was carried out as described above, except that the first antibody (2.5.2, purified as described above) was diluted to 1 μg/ml in StartingBlock. Results are shown in FIG. 2. The antibody specifically detected tau isoforms phosphorylated at Ser422 in the RAB-insoluble/SDS-soluble brain extracts. This is to be expected since hyperphosphorylated tau usually occurs in this fraction of the extract. Normal tau occurs in the RAB-soluble fraction and clearly there is no cross-reaction of the antibody with this fraction in any of the samples. The intensity of staining correlates with disease severity and no staining is evident with the control brain extract.

### Example 6

### Immunohistochemical Analysis of AD Brain Sections

Cryostat sections of unfixed brain tissue from cortical human brain regions, obtained postmortem from a patient that was positively diagnosed for Alzheimer's disease, Braak stage VI.

Sections were labeled by indirect immunofluorescence with anti-p-tauS422A antibody (Wheatley S. and Wang Y., Methods Cell Bio 57 (1998) 313-332). A successive two-step incubation was used to detect bound anti-p-tauS422A antibodies, which are revealed by affinity-purified goat anti-mouse (GAM) IgG (H+L) conjugated to Alexa 488 (Molecular Probes). A counterstaining against Aβ peptide was doe to reveal amyloid-β plaques.

In detail, Sections were cut at -18° C. using a cryostat (Leica, CM 3050 S) at a nominal thickness of 10 μm. After adhesion to pre-cooled glass slides (Super Frost Plus, Menzel, Germany), sections were hydrated in PBS and treated with 100% acetone pre-cooled at -20° C. for 2 min. Washing with PBS was done twice for two minutes. Blocking of unspecific binding sites was done by incubation in PBS with 1% bovine serum albumin (BSA) and 1% ovalbumin (OVA) and 1% normal goat serum for 20 min. Anti-p-tauS422A antibodies were used at a concentration of 10 μg/ml in PBS with 1% BSA, 1% OVA and 1% normal goat serum for 1 hour. After washing with PBS and 1% BSA, slides were incubated with affinity-purified goat anti-mouse (GAM) IgG (H+L) conjugated to Alexa 488 (Molecular Probes) at 15 ug/ml in PBS with 1% BSA for 1 hour. Slides were washed 3×5 minutes with PBS and 1% BSA and counterstained with a monoclonal mouse antibody against Aβ (BAP-2, Dr. M. Brockhaus, F. Hoffmann La Roche, EP130424) at 5 μg/ml in PBS with 1% BSA for 1 hour. Slides were washed 3×5 minutes with PBS rinsed with water and immersed in 0.3% sudan black in 70% aqueous ethanol for 5 minutes to reduce autofluorescence of lipofuscin. After one rinse with 70% ethanol and 2 rinses with water slides were washed with 2×3 minutes PBS and embedded with fluorescence mounting medium (S3023 Dako). Controls included unrelated mouse IgG1 antibodies (Sigma) and the secondary antibody alone, which all gave negative results.

Images were recorded with a Zeiss Axioplan2 using a 10×/0.3 objective. Images of both recorded fluorescence channels were merged with Photoshop.

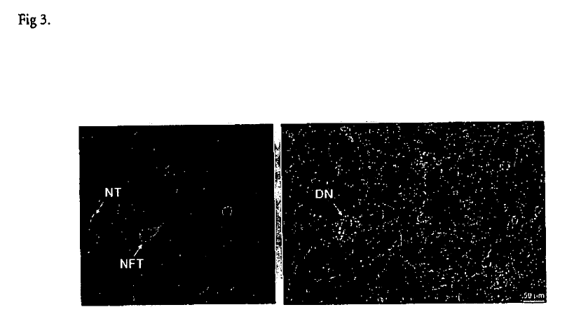

Results are shown in FIG. 3. The antibody stained typical structures associated with the pathology of Alzheimer's Disease, the most prominent of these being neurofibrillary tangles. Numerous neuropil threads were also evident, as were dystrophic neurites surrounding plaques (visualised by counter staining with an amyloid-specific antibody).

### Example 7

### Western blots for detecting pSer422 in tau and MAP2 in neuroblastoma cell extracts

The peptide immunogen used to generate the monoclonal antibodies is based on the amino acid sequence immediately surrounding S422 in tau, namely S416-IDMVDSPQLATLA-D430. This sequence occurs in tau towards its C-terminus. A homology search of protein sequence databases revealed that the highly homologous amino acid sequence S1802-INLLESPQLATLA-E1816 occurs towards the C-terminus of the microtubule-associated protein MAP2. LAN-5 cells treated with okadaic acid and analyzed by western blot using the antibodies described above do indeed contain a high molecular weight protein which cross-reacts with antibodies 2.5.2 and 2.20.4. The molecular weight of this protein is consistent with it being MAP2.

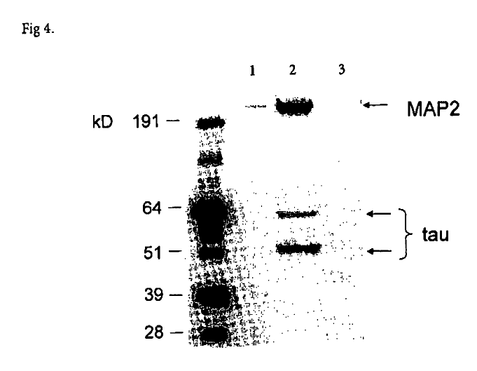

Cells (1×10⁷/well in serum-free medium) were treated with DMSO or 10 μM K252a (Alexis) for 1 hr at 37° C.; the medium was then supplemented with DMSO or 2 μM okadaic acid for a further 1 hr at 37° C. Medium was removed and the cells extracted into 100 μl of Cytobuster (Novagen) and 25 μl used for western blot analysis. Western blots were performed as described in example 5. The result is shown in FIG. 4. The antibody 2.5.2 showed cross-reactivity with phosphorylated MAP2.

### Example 8 =cl ELISA assay for detecting tau/pSer422 in okadaic acid-tereated neuroblastoma cells

LAN-5 neuroblastoma cells express tau endogenously. Phosphorylation of endogenous tau at Ser422 occurs when the cells are treated with okadaic acid.

LAN-5 neuroblastoma cells are cultured in medium at 37° C. and plated into 96-well microtitre plates at a cell density of 2.5×10⁵ cells/well in 100μl of serum-free medium. After 24 h in culture the cells are then treated with 2.5 μM okadaic acid for 2 h. Subsequently, 10 μl of a solution comprising 1 mg/ml digitonin, 10 mM EDTA is added and the cells shaken for 30 min at 4° C. This extract is then used directly in the ELISA assay described below.

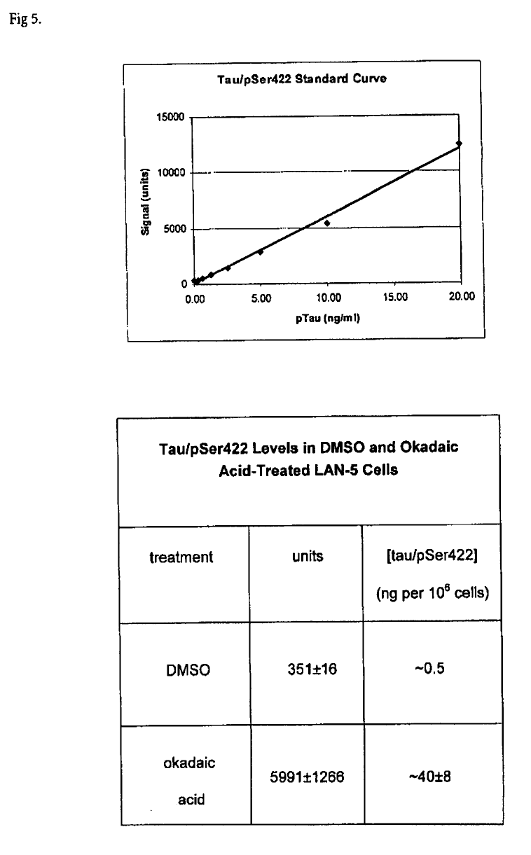

Antibody 2.5.2 was biotinylated using biotin-NHS at a ratio of 20:1 in phosphate buffered saline, pH7.2. Antibody 5A6 (Developmental Studies Hybridoma Bank, University of Iowa) was labelled with BV-TAG™ at a ratio of 8:1 according to the protocol supplied by BioVeris (BioVeris Corporation, Gaithersburg, Md.). In a typical assay, 25 μl streptavidin-coated magnetic beads (Dynal/Invitrogen Corporation) at a dilution of 1:50 was preincubated for 30 min with 25 μl of 1 μg/ml biotin-antibody at RT. To this mixture was then added 50 μl of cell extract and 25 μl BV-tagged antibody 2.5.2 (prepared as described above). The mixture was then incubated for 3 h at 4° C. with shaking. All samples were prepared in 96-well microtitre plates. Subsequently 125 ul buffer was added to each sample and plate measured in BioVeris M384 analyser. Tau/pSer422 standard curve was prepared by diluting ERK2- or P38-phosphorylated tau in cell extraction buffer; calculation of tau/pSer422 level in cells assumes 1 mole phospho-Ser422 phosphate/mole tau for the phospho-tau standard. FIG. 5 shows a standard curve and typical values obtained (on the basis of 1×10⁶ cells) before and after okadaic acid treatment.

1. An antibody or a fragment thereof which binds to an epitope consisting of Ser-Ile-A1-A2-A3- A4-Ser (PO₃H₂)-Pro-Gln-Leu-Ala-Thr-Leu-Ala-A5 (SEQ ID NO: 9), and does not bind to an epitope consisting of Ser-Ile-A1-A2-A3- A4-Ser-Pro-Gln-Leu-Ala-Thr-Leu-Ala-A5 (SEQ ID NO: 8), wherein Al is Asp or Asn, A2 is Met or Leu, A3 is Val or Leu, A4 is Asp or Glu and A5 is Asp or Glu.

2. The antibody of claim 1, wherein A1 is Asp, A2 is Met, A3 is Val, A4 is Asp and A5 is Asp.

3. The antibody of claim 2, wherein its Kd for the peptide consisting of SEQ ID NO: 9 is lower than 100nM.

4. The antibody of claim 1, wherein A1 is Asn, A2 is Leu, A3 is Leu, A4 is Glu and A5 is Glu.

5. The antibody of claim 1, wherein the antibody binds to tau comprising phospho-Ser422 and does not bind to tau comprising unphosphorylated Ser422.

6. The antibody according to claim 1, wherein the antibody binds to MAP2 comprising phospho-Ser1808 and does not bind to MAP2 comprising unphosphorylated Ser1808.

7. The antibody according to claim 1, wherein the antibody is monoclonal antibody.

8. The antibody according to claim 1, wherein the antibody is produced by the hybridoma DSM ACC2762 or DSM ACC2763.

9. A method for diagnosing a neurological disorder, which comprises
(a) contacting a biological sample with the antibody of claim 1; and
(b) detecting a complex formed between the antibody and tau or MAP2.

10. The method of claim 9, wherein the neurological disorder is Alzheimer's disease.

11. A method for detecting tau comprising phospho-Ser422 in a biological sample, which comprises
(a) contacting a biological sample with the antibody of claim 1; and
(b) detecting a complex formed between the antibody and tau.

12. The method of claim 11, wherein the complex is detected by western blotting, immunohistochemistry or ELISA.

13. A method for detecting MAP2 comprising phospho-Ser1808 in a biological sample, which comprises
(a) contacting a biological sample with the antibody of claim 1; and
(b) detecting a complex formed between the antibody and MAP2.

14. The method of claim 12, wherein the complex is detected by western blotting, immunohistochemistry or ELISA.

15. A kit for diagnosing a neurological disorder, which comprises the antibody according to claim 1.

16. A kit of claim 15, wherein the neurological disorder is Alzheimer's disease.

17. A kit for detecting tau comprising phosho-Ser422, which comprises the antibody according to claim 1.

18. A kit for detecting MAP2 comprising phospho-Ser1808, which comprises the antibody according to claim 1.

19. A cell which produces an antibody according to claim 1.

20. The cell of claim 19, which is a hybridoma.

21. The cell of claim 20, wherein the hybridoma is DSM ACC2762 or DSM ACC2763.
